(19) United States
(12) Patent Application Publication
(10) Pub. No.: US 2015/0276205 A1
(43) Pub. Date: Oct. 1, 2015

(54) LIGHTING FIXTURE WITH ANTIMICROBIAL/ANTIFUNGAL SHEET AND CLEAN ROOM CAPABILITY

(71) Applicant: GE LIGHTING SOLUTIONS LLC, East Cleveland, OH (US)

(72) Inventors: Dengke Cai, Willoughby, OH (US); Matthew A. Bugenske, Shaker Heights, OH (US); Sebastien Magnan, Montreal QC (CA); Francois Turgeon, St-Eustache QC (CA); Jean-Francois Richard, St-Hubert QC (CA); Angela Wong, Dorval QC (CA)

(73) Assignee: GE LIGHTING SOLUTIONS LLC, East Cleveland, OH (US)

(21) Appl. No.: 14/227,477

(22) Filed: Mar. 27, 2014

(51) Int. Cl. F21V 33/00; F21S 8/04; A61L 9/14; B32B 37/14; B32B 37/24; F21V 21/00; F16J 15/02

(52) U.S. Cl. CPC F21V 33/0064; F21V 21/00; F21S 8/04; F16J 15/02; B32B 37/144; B32B 37/24; A61L 9/14; B32B 2037/243; B32B 2255/00; B32B 2307/7145; A61L 2209/12

(57) ABSTRACT

A system and method according to various embodiments can include a lighting fixture comprising a light source. An antimicrobial additive is added to an outer light emitting surface of the lighting fixture exposed to air. A sealing substrate is positioned between the antimicrobial additive and the light source to provide clean room capabilities when the lighting fixture is installed to seal a plenum.

### I. FIELD OF THE INVENTION

The present invention relates generally to the field of antimicrobial lighting fixtures. More particularly, the present invention relates to reducing bacterial growth, resisting bio-adhesion of microbes, and providing clean room capability, for example, in a healthcare facility.

### II. BACKGROUND OF THE INVENTION

A clean room is a controlled environment in which the concentration of airborne particles is controlled to specified limits. Airborne contamination must be continually removed from the air. The level to which these particles need to be removed depends upon the standards required.

Clean room environments are of immense value in many industries, including healthcare, aerospace, medical device production, semiconductors, and pharmaceutical. The low density of environmental pollutants such as airborne microbes, bacteria, particles, and dust within these clean room facilities reduces the amount of contamination within these facilities.

The only way to control contamination is to control the total environment. Eliminating airborne contamination is really a process of control. These contaminants are generated by people, process, facilities and equipment. For example, in the healthcare industry, it is estimated that between 5% and 10% of patients admitted to hospitals acquire one or more healthcare-associated infections, which leads to more than a million people worldwide being affected by infections acquired in hospitals. Health-care associated infections are also an important problem in extended care facilities, including nursing homes and rehabilitations units. These health-care acquired infections are associated with nearly 100,000 deaths annularly.

Patients infected with healthcare-associated microbes frequently contaminate items in their immediate vicinity with microbes that may remain viable on surfaces for days to weeks. Contaminated surfaces in healthcare facilities contribute to the spread of healthcare-associated microbes. In some instances, patients acquire microbes following direct contact with contaminated equipment or other surfaces. Contaminated surfaces can act as sources from which healthcare workers contaminate their hands. Healthcare workers can contaminate their hands by touching contaminated surfaces, and can transmit microbes if their hands are not cleansed appropriately.

Another critical source of contamination is inadequate cleaning of rooms after discharging a patient with certain contagious diseases, which puts subsequent patients admitted to the room at risk of acquiring the organism. Routine cleaning of patient rooms is often below the required standard. Therefore, improved cleaning and disinfection of the environment can reduce the risk of patients acquiring multi-drug resistant microbes. Cleaning, disinfecting and sterilization save lives and improve patient outcomes. Providing patients with a safe environment of care requires appropriate cleaning and disinfection of medical equipment and environmental surfaces.

Furthermore, many microbes can form multicellular coatings, called biofilms. Biofilms are any group of microorganisms in which cells stick to each other on a surface. Biofilms can facilitate the proliferation and transmission of microorganisms by providing a stable protective environment. The biofilm colonizes by attaching to a surface or host, growing and multiplying. Biofilms can be prevalent in facilities such as hospitals, schools, public restrooms, restaurants, bars, club houses, and daycare centers.

Accordingly, much research has been devoted toward preventing colonization of microbes on the surfaces in such facilities, especially healthcare facilities, and preventing growth of bacteria by the use of antimicrobial agents. Conventional techniques employed in the lighting industry to reduce bacterial growth and maintain a sanitary environment include, for example, antimicrobial doped powder coating or paint to coat the metal bezels of lighting fixtures and blended antimicrobial additives incorporated into plastic components of the lighting fixtures. However, these technologies are limited by EPA regulation on the doping ratio of antimicrobial additives.

### III. SUMMARY OF EMBODIMENTS OF THE INVENTION

Given the aforementioned deficiencies, a need exists for a lighting system and method that provides antimicrobial/antifungal to control microbial growth over the entire illuminating surface area of a lighting fixture. There also remains a need for a lighting system and method that provides clean room capability. There remains a further need for a clean room and controlled environment facility having the ability to control bacterial growth through the use of a ceiling light, which delivers a pleasant, uniform light to illuminate a room.

A system according to various exemplary embodiments can include a lighting fixture comprising a light source. An antimicrobial additive is added to an outer light emitting surface of the lighting fixture exposed to air. A sealing substrate is positioned between the antimicrobial additive and the light source to provide clean room capabilities when the lighting fixture is installed to seal a plenum.

A method of using a lighting system according to various exemplary embodiments can include adding an antimicrobial additive to an outer light emitting surface of a lighting fixture configured to be exposed to air; and sealing a plenum with a sealing substrate provided between the antimicrobial additive and a light source of the lighting fixture when the lighting fixture is installed.

Further features and advantages of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art(s) based on the teachings contained herein.

### IV. BRIEF DESCRIPTION OF THE DRAWINGS

FIG. 1 is an exploded view of an exemplary lighting system in accordance with the present teachings;

FIG. 2 is an exploded view of an exemplary cover plate assembly in accordance with the present teachings; (need to be reedited)

FIG. 3 is a partial perspective view of an interior region of an exemplary lighting system in accordance with the present teachings;

FIG. 4 is a view of an exemplary embodiment of a lighting system installed within a ceiling member according to the present teachings;

FIG. 5 is a view of an exemplary embodiment of a lighting housing in accordance with the present teachings; and

FIG. 6 is a flowchart of an exemplary method of practicing the present invention in accordance with the present teachings.

The present invention may take form in various components and arrangements of components, and in various process operations and arrangements of process operations. The present invention is illustrated in the accompanying drawings, throughout which, like reference numerals may indicate corresponding or similar parts in the various figures. The drawings are only for purposes of illustrating preferred embodiments and are not to be construed as limiting the disclosure. Given the following enabling description of the drawings, the novel aspects of the present invention should become evident to a person of ordinary skill in the art.

### V. DETAILED DESCRIPTION OF VARIOUS EMBODIMENTS

The following detailed description is merely exemplary in nature and is not intended to limit the applications and uses disclosed herein. Further, there is no intention to be bound by any theory presented in the preceding background or summary or the following detailed description.

Throughout the application, description of various embodiments may use “comprising” language, however, it will be understood by one of skill in the art, that in some specific instances, an embodiment can alternatively be described using the language “consisting essentially of” or “consisting of”.

For purposes of better understanding the present teachings and in no way limit the scope of the teachings, it will be clear to one of skill in the art that the use of the singular includes the plural unless specifically stated otherwise. Therefore, the terms “a,” “an” and “at least one” are used interchangeably in this application.

Unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. In some instances, “about” can be understood to mean a given value ±5%. Therefore, for example, about 100 nm, could mean 95-105 nm. At the very least, each numerical parameter should at least be construed in light of the number of reported significant digits and by applying ordinary rounding techniques.

Various embodiments provide a system and method that relates to antimicrobial function for lighting system and components. Various embodiments relate to a method and system comprising a lighting fixture with a film on the light emitting side. The film acquires antimicrobial/antifungal properties through antimicrobial compounds applied to the surface or internally blended antimicrobial compounds. In various embodiments, the antimicrobial film is laminated onto a clear or translucent substrate, which is positioned between the lighting source within the lighting fixture and the light emitting area.

Various embodiments relate to a system and method that provides lighting devices with effective antimicrobial activity in order to reduce the growth of bacteria, and provides a completely sealed housing. Various embodiments provide a clean room and controlled environment facility having the ability to control bacterial growth through the use of a ceiling light, which delivers a pleasant, uniform light to illuminate a room. Various embodiments relate to a lighting system and method that exhibits antimicrobial/antifungal properties to control microbial growth and reduce microbe colonization over the entire outer illuminating surface area exposed to air.

In various embodiments, a lighting system and method provides clean room capability. In various embodiments, a laminated clear/translucent plate is installed in a ceiling grid such that it acts a particle barrier that seals the overhead plenum space from the light emitting area and provides a lighting fixture with clean room capabilities.

Various embodiments provide a lighting system and method for luminaires used in controlled environments such as hospitals, nursing homes, hotels, schools, food processing facilities, professional lighting, swimming facilities, agricultural facilities, pools, etc. where it is desirable to mitigate or control the growth of microbes. Thus, the light system and method provides good visibility and contamination control.

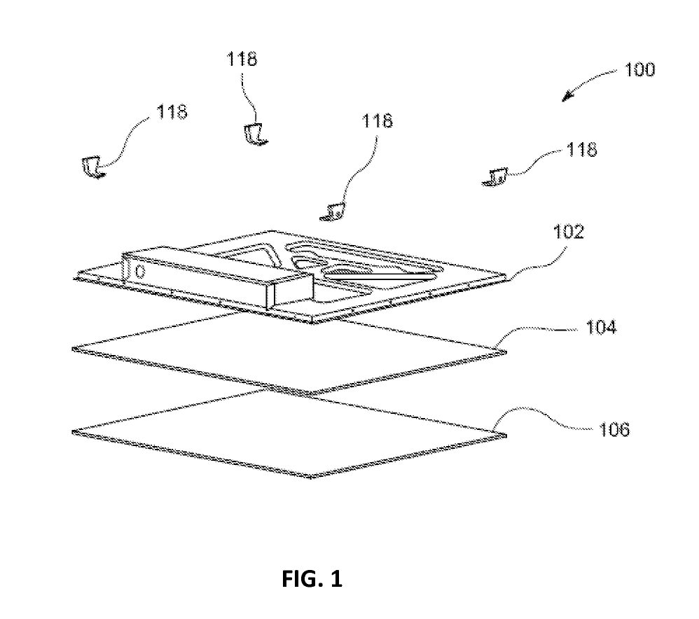

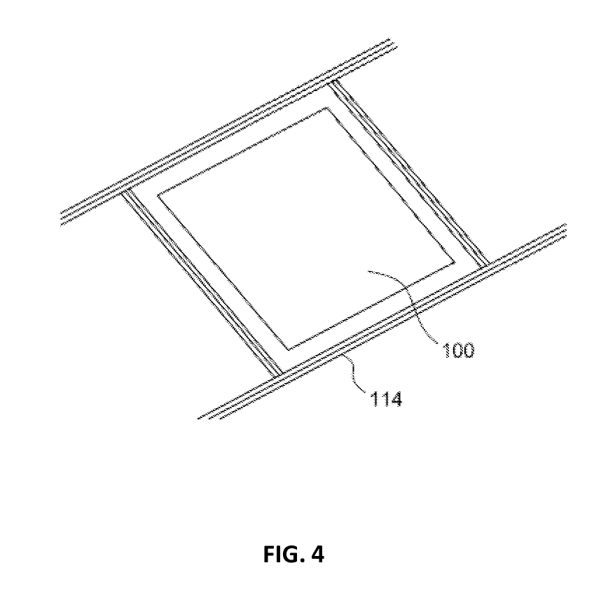

An exemplary embodiment of a lighting fixture 100 or troffer for directing light emitted from a light source toward an area to be illuminated is shown in FIGS. 1 and 4. The lighting fixture 100 can be used to provide antimicrobial/antifungal capabilities to reduce the growth of microbes and resist bio-adhesion over the entire illuminating surface area. The lighting fixture 100 can also provide a controlled area with clean room capabilities such that the plenum space is totally sealed to protect against airborne microbes, as shown in FIG. 4.

As shown in FIG. 1, the lighting fixture 100 may be formed by combining a light source 102 with a cover plate assembly 106. An attachment mechanism, such as double-side tape 104, is attached between the light source 102 and the cover plate assembly 106.

The lighting fixture 100 can include a light source, such as an LED luminaire 102. In general, the luminaire 102 is a complete lighting unit consisting of a single or multiple lamps together with the parts designed to distribute the light, to position and protect the lamps, and to connect and interface the lamps to the power source. The details of the components of the luminaire will not be described herein, because it is not the subject of the invention.

An example of an LED luminaire 102, which may be used in the present teachings, is an ET22 Luminaire available from General Electric. In the “ET22” product name, the “E” stands for “edge lighting” and the “T” stands for “troffer.” The number 22 represents the fixture type having dimensions 2′×2′ (605×605 mm). In lieu of or in addition to luminaires, any light source can be used to emit light from the lighting fixture 100. Those skilled in the art would recognize various mechanisms for emitting light from the lighting fixture 100.

In FIG. 1, LED luminaire 102 can be coated with an adhesive layer 104 via a bonding method. The adhesive layer 104 can be, for example, a double-sided tape 104, which provides a mechanism for attaching or bonding the cover plate assembly 106 to the LED luminaire 102. The double-sided tape 104 may be respectively attached to facing surfaces of the LED luminaire 102 and the cover plate assembly 106.

The cover plate assembly 106 and the LED luminaire 102 may thus be combined to form the lighting fixture 100. The double-sided tape 104 may be attached to the surface of the front bezel of the LED luminaire 102. For example, four pieces of the double-sided tape having a thickness of approximately ¼″ may be employed. However, the size and number of pieces of the double-sized tape 104 may vary.

In an exemplary embodiment, the double-sided tape 104 in which adhesives are formed on both sides of a supporting layer may be a bonding tape made by 3M™ Corporation. The double-side tape 104 has a product name VHB™ and is made of foam.

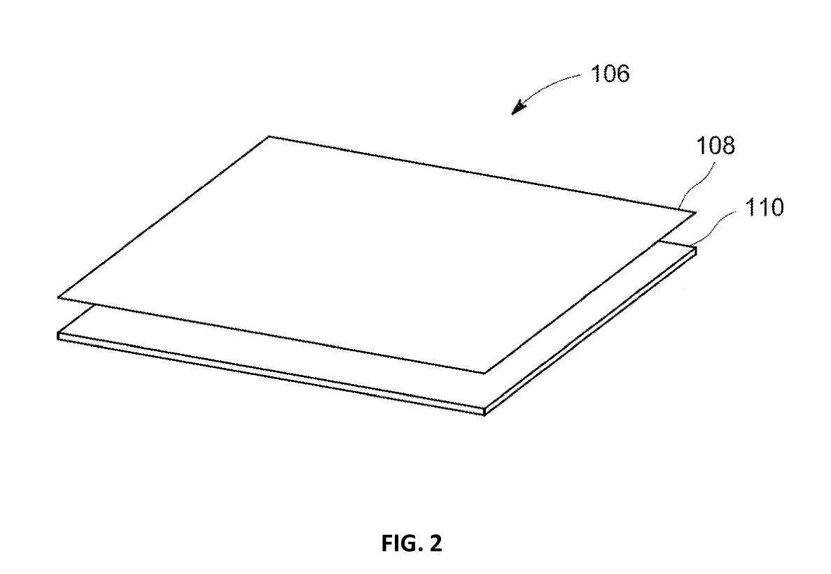

As shown in FIG. 2, the cover plate assembly 106 may include several stacked layers comprising an antimicrobial film 108, a substrate 110. The antimicrobial film 108 functions as an outer film, which is positioned on the front side between the lighting fixture and the illuminated area. With the antimicrobial film 108 on the front side of the lighting fixture exposed to the air, the antimicrobial film 108 provides antimicrobial/antifungal properties released through surface coated or integrally blended antimicrobial compounds. Namely, the front side antimicrobial film 108 provides antimicrobial/antifungal properties derived through top coatings or impregnated antimicrobial/antifungal compounds within the film 108.

A blended antimicrobial additive may be coated onto a transparent plastic film having a thickness varying from less than 1 um to few mm. The antimicrobial film 108 may be manufactured having a flexible film structure comprising an antimicrobial agent incorporated into the manufacture of a plastic film. In some embodiments, the flexible antimicrobial film may be a single layer film comprising an antimicrobial agent incorporated into the manufacture of a plastic film. In other embodiments, the flexible antimicrobial film may consist of multiple layer films including one or more layer films comprising an antimicrobial agent incorporated into the manufacture of a plastic film and wherein the antimicrobial layer is positioned as an outer layer. Using plastic permits a wide variety of shapes to be easily manufactured.

There are several different methods of making the antimicrobial film 108 with antimicrobial additives coated on the outer surface of a substance or with the antimicrobial additives blended within a substance. The most common method is to blend antimicrobial additive into plastic or another substance and then form parts by injection molding. Another method is to coat antimicrobial coatings with or without binder onto plastic or another substance.

An example of a suitable antimicrobial agent that may be incorporated into a substance, such as plastic, according to the present teaching is exemplified by but not limited to silver (Ag) and Ag doped materials. The most common antimicrobial being incorporated into materials is silver and Ag doped materials. Silver is a powerful, natural antibiotic and is one of the oldest antimicrobial agents on record. Silver derives its broad spectrum antimicrobial activity from the ability of silver ions. Silver ions released from the antimicrobial agent, come in contact with microbes and the microbes are inhibited and destroyed.

Thus, under humidity the antimicrobial agent releases silver ion in the air to effectively kill or control microorganisms in the air. Thus in such an exemplary embodiment of the present teaching employing silver, the outer illuminating surface area containing silver is capable of releasing silver ions to create an effective bacterial barrier and inactivating a wide range of microbes.

It should be understood that the term “antimicrobial additive” as used throughout the disclosure means any chemical additive that reduces the level of bacteria, molds, fungi and other microbes and are commonly practiced as additives supplied directly into plastic materials, coatings, paints, etc. In various embodiments, one or more suitable antimicrobial additives can be selected from the following group: Ag, zinc and copper etc., and ions doped carriers such as zeolite, glass and some types of organic hosts, silver nano particles, tricolsan, and quartenary ammonium component, etc. This list is merely exemplary and is not exclusive.

An “antimicrobial coating”, as used herein, refers to any coating or paint or surface grown layer that has antimicrobial function that can be applied to the surface of a device or component. Antimicrobial properties can be derived from the above mentioned antimicrobial additives blended within or applied as a coating itself, like TiO2, etc.

An “antimicrobial agent”, as used herein, refers to a chemical that is capable of decreasing or eliminating or inhibiting the growth of microbes such a known in the art. The antimicrobial agent can be antimicrobial additive blended chemicals, an antimicrobial additive used alone, or any precursors that initiates an antimicrobial function after further reactions and processes, like crosslinking, crystallizing and polymerization etc.

While a number of methods of manufacturing an antimicrobial film have been exemplified herein, it is understood that any antimicrobial film, which meets the requirement of controlling the growth of microbes and/or reducing microbial colonization may be suitable for use in the present invention. The choice of a particular antimicrobial film 108 may depend on the extent of microbial growth present. Those skilled in the art are well aware as how to select one or more antimicrobial film for a given treatment environment. For example in a hospital setting, the antimicrobial film may be Ag doped particles blended containing film or anatase TiOx film etc.

After the antimicrobial film is prepared with the blended antimicrobial compound, the antimicrobial film 108 can be laminated onto substrate 110, as shown in FIG. 2. The antimicrobial film 108 is affixed to the outside surface of the substrate 110 such that the antimicrobial film layer is exposed to the air. For example, the antimicrobial film 108 can be formed as a sheet that covers the entire surface of the substrate. This enables the antimicrobial film to exhibit antimicrobial properties to reduce the growth of microbes and reduce microbial colonization over the entire outer light illuminating surface area.

In various embodiments, the substrate 110 may consist of a clear/translucent substrate, which is substantially flat. The clear/translucent substrate can function as a cover plate that provides a mechanical support for the flexible antimicrobial film 108 applied thereon. The clear/translucent substrate 110 acts as a mechanical holder for the flexible antimicrobial sheet 108 and enable its integration into the lighting system 100.

The clear/translucent substrate 110 can be formed of a variety of materials. Suitable substrate such as PMMA, PET, PC, glass formed having a thickness between 1 mm to 3 mm.

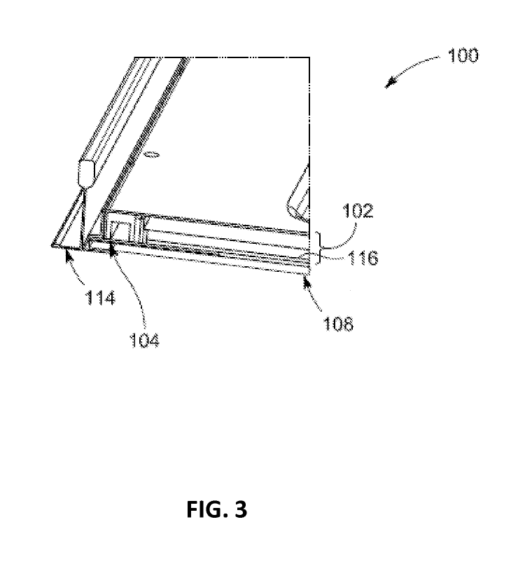

After the antimicrobial film 108, the substrate 110, are assembled to form the cover plate assembly 106, as shown in FIG. 1. Once the lighting fixture 100 is assembly, the lighting fixture 100 can be installed, for example, into a ceiling grid or wall within a room. The example in FIG. 3 depicts the lighting fixture 100 as a recessed lighting unit, installed within a ceiling grid 114.

In FIG. 3, the lighting fixture 100 includes a luminaire 102 configured as two feet wide by two feet long with a single array of edge lighting LEDs 116 extending along and edge of the lighting fixture 100. The antimicrobial plate 106 is affixed to the bezel of the luminaire 102 with the use of double-sided adhesive tape 104. The antimicrobial film 108 is attached to the front side or outer surface of the lighting fixture exposed to the air to provide antimicrobial/antifungal properties to reduce growth of microbes and inhibit microbial colonization.

The assembled lighting fixture 100 also provides clean room capabilities by totally sealing the installed device to maintain ceiling integrity and protect against airborne microbes and particle infiltration. As shown in FIG. 4, the light fixture 100 is installed such that the clear/translucent plastic substrate 110 laminated with the antimicrobial film 108 is installed in the ceiling grid and completely seals the plenum space from the area of the light emitting side. Thus, both the clear/translucent substrate and the antimicrobial film 108 are installed within the ceiling grid 114. This installation configuration serves to provide a particle barrier that seals the plenum space, provide the lighting fixture with clean room compatibilities, and provide the exterior surface of the lighting fixture with antimicrobial/antifungal capabilities to reduce the growth of microbes.

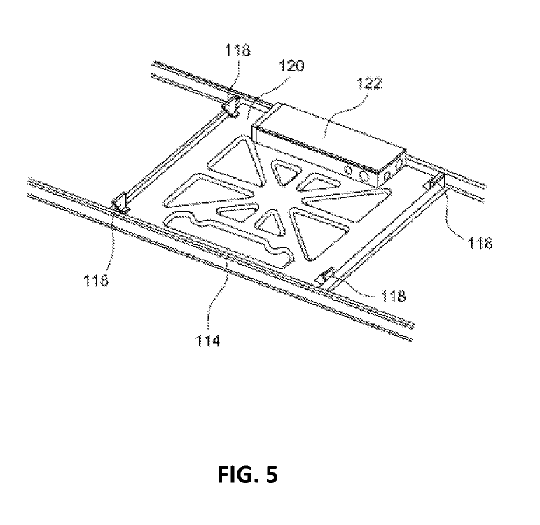

As illustrated in FIGS. 4-5, the lighting fixture is mounted to the ceiling grid by using mounting clips 118 as shown, along with a frame portion 120, and a container for housing electronics 122. With the antimicrobial plate 106 installed on the lighting fixture (antimicrobial film on the room side) and the lighting fixture installed into the ceiling grid as shown in FIG. 4, the fixture and the plate becomes a barrier that seals dust from entering the room below. Therefore once the fixture and plate are installed, the room side is sealed from the dusty plenum above.

Although the lighting system is illustrated and described with respect to an overhead plenum within a ceiling, it should be understood by that the lighting fixture may be installed within any plenum requiring clean room capabilities. For example, the lighting fixture may be installed within a plenum of a dashboard of a mobile medical testing vehicle.

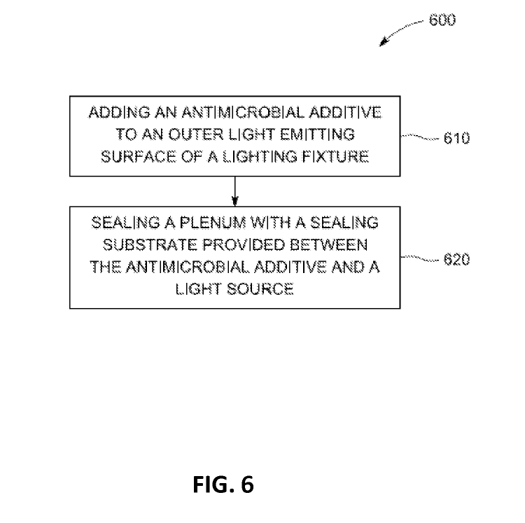

FIG. 6 is a flowchart of an exemplary method 600 of practicing an embodiment of the present teachings. A method of providing a lighting system with enhanced antimicrobial properties, bio-adhesion resistance, and clean room capabilities is described herein. In Step 610, a blended antimicrobial compound is added on the front side or outer surface of a lighting fixture to reduce bacterial growth and control microbial colonization over the entire light emitting outer surface area. In Step 620 of the exemplary method, the light fixture is installed to seal the plenum space from the light emitting outer surface area to provide clean room capability.

In general, the present teaching relates to a system and method that provide a lighting fixture exhibiting antimicrobial/antifungal capabilities over the entire light emitting area exposed to the air. In use, when the light fixture is activated the light contacts the antimicrobial compound causing the release of antimicrobial agents to combat airborne microbes and fungi. Also, the plenum is totally sealed when the lighting fixture is installed providing the lighting fixture with clean room compatibility.

Use of the same device and technology can be transferred to different product lines by changing the size of the laminated plate. Although the exemplary embodiment is depicted having a substantially rectangular shaped geometry, alternative embodiments of the device can be configured to have any number of shapes. Those skilled in the art would understand that various sizes, shapes and configurations may be envisioned for the device without departing from the scope of the invention.

Furthermore, the present teaching is not limited to medical settings. The present teaching is applicable in other industrial applications where the control of the growth of microbes and the reduction of microbial colonization are desired. In addition to a hospital setting, some of the other applications of the antimicrobial lighting fixture 100 include, for example, nursing homes, hotels, schools, food processing facilities, agricultural facilities, pools, medical devices production, pharmaceutical packaging, and research and development facilities.

Testing was conducted for a lighting fixture comprising an antimicrobial/antifungal film prepared according to the present teaching regarding the proliferation of microbes and the viability of the microbes. The proliferation and the viability of the microbes were tested with the JIS Z 2801 test method. The JIS Z 2801 test method is designed to quantitatively test the ability of plastics and other antimicrobial surfaces to inhibit the growth of microorganisms or kill them over a 24 hour period of contact.

The test results showed continuous inhibition of microbe growth for microorganisms, such as Staphylococcus aureus, Escherichia coli, Klebsiella pneumonia, MRSA Staphylococcus aureus, Acinebacter baumanii, Candida albicans, Bacillus cereus, Aspergillus niger and Streptococcus pneumoniae. These experiments were repeated several times with the same results. Thus, it is clearly evident that the lighting fixtures prepared according to the invention are effective antimicrobial agents. According to the test, an antibacterial product is determined to have antibacterial effectiveness when the antibacterial activity is greater than or equal to 99%.

Alternative embodiments, examples, and modifications which would still be encompassed by the disclosure may be made by those skilled in the art, particularly in light of the foregoing teachings. Further, it should be understood that the terminology used to describe the disclosure is intended to be in the nature of words of description rather than of limitation.

Those skilled in the art will also appreciate that various adaptations and modifications of the preferred and alternative embodiments described above can be configured without departing from the scope and spirit of the disclosure. Therefore, it is to be understood that, within the scope of the appended claims, the disclosure may be practiced other than as specifically described herein.

We claim:

1. A lighting system comprising:
a lighting fixture comprising a light source; and
a sealing substrate with antimicrobial additive to provide clean room capabilities when the lighting fixture is installed to seal a plenum.

2. The system according to claim 1, wherein the antimicrobial additive is configured as a sheet affixed to the outer light emitting surface.

3. The system according to claim 2, wherein the antimicrobial additive is incorporated within the sheet by a blending process.

4. The system according to claim 2, wherein the antimicrobial additive is coated onto the sheet by a coating process.

5. The system according to claim 2, wherein the sheet comprises a transparent/translucent plastic film.

6. The system according to claim 1, wherein the antimicrobial additive is laminated onto the sealing substrate.

7. The system according to claim 6, wherein the sealing substrate is a transparent/translucent plastic substrate.

8. The system according to claim 6, wherein the plenum comprises an overhead plenum above a ceiling grid.

9. The system according to claim 8, wherein the sealing substrate laminated with the antimicrobial additive is installed within the ceiling grid such that the overhead plenum is completely sealed.

10. The system according to claim 1, wherein the entire outer light emitting surface exposed to the air exhibits antimicrobial properties; and
wherein a plenum is sealed to a ceiling grid to provide clean room capabilities.

11. A method of use of a lighting system, comprising sealing a plenum with a sealing substrate provided between an antimicrobial additive and a light source of the lighting fixture when the lighting fixture is installed.

12. The method according to claim 12, further comprising affixing a sheet comprising the antimicrobial additive to the outer light emitting surface.

13. The method according to claim 13, further comprising incorporating the antimicrobial additive within the sheet by a blending process.

14. The method according to claim 13, further comprising coating the antimicrobial additive onto the sheet.

15. The method according to claim 12, further comprising laminating the antimicrobial additive onto the sealing substrate.

16. The method according to claim 16, further comprising installing the sealing substrate laminated with the antimicrobial additive within a ceiling grid such that an overhead plenum is completely sealed.

17. The method according to claim 12, wherein the entire outer light emitting surface exposed to the air exhibits antimicrobial properties and clean room capabilities; and
wherein a plenum is sealed to a ceiling grid to provide clean room capabilities.
